(19) United States
(12) Patent Application Publication
(10) Pub. No.: US 2012/0285538 A1
(43) Pub. Date: Nov. 15, 2012

(54) DYE COMPOUND, DYE-SENSITIZED SOLAR CELL USING THE SAME AND DYE SOLUTION

(75) Inventors: Wei-Cheng Tang, Taoyuan (TW); Szu-Yu Lin, Taoyuan (TW)

(73) Assignee: EVERLIGHT USA, INC., Pineville, NC (US)

(21) Appl. No.: 13/180,681

(22) Filed: Jul. 12, 2011

(30) Foreign Application Priority Data
May 9, 2011 (TW) 100116099

(51) Int. Cl. H01L 51/46; C07D 211/06; C09B 57/00; C07C 255/43

(52) U.S. Cl. 136/263; 558/401; 546/203; 252/182.12

(57) ABSTRACT

The present invention provides a dye compound of formula (I) or a salt thereof and a dye-sensitized solar cell using the dye compound:

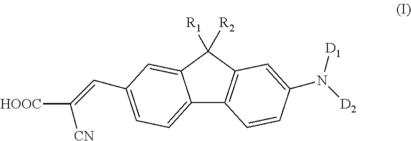

wherein R₁ and R₂ are, independently, H, C₁-C₁₂alkoxy or a halogen; and D₁ and D₂ are, independently, C₁-C₁₂alkyl, or D1, D2 and N form one of

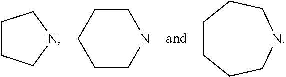

The dye compound is applicable to a dye-sensitized solar cell for increasing photoelectric efficiency of the dye-sensitized solar cell.

### FIELD OF INVENTION

The present invention relates to dye compounds and photoelectric elements using the dye compounds, and more particularly, to a dye compound and a dye-sensitized solar cell using the dye compound.

### BACKGROUND OF THE INVENTION

It is currently important to convert solar energy into electric energy of a solar cell to alleviate energy crisis and environmental pollution in the world. It is a trend to develop a dye-sensitized solar cell due to low cost, large area, flexibility, and transparency, which may be used on architecture.

Grätzel et al. have published literatures about dye-sensitized solar cells to demonstrate usage of the dye-sensitized solar cells (for example, O'Regan, B.; Grätzel, M. Nature 1991, 353, 737). Generally, a dye-sensitized solar cell includes anode/cathode electrodes, nano titanium dioxide, a dye, and an electrolyte; among them, the dye is most critical to cell efficiency.

Currently, a ruthenium complex is a photo-sensitized dye with higher photoelectric conversion efficiency, but has issues such as high cost to manufacture and low supply to meet demand. Organic-sensitized dyes have high molar extinction coefficients and are flexible for molecular design, such that dye-sensitized solar cells with various colors may be formed for various applications. Recently, coumarin (Hara, K.; Sayama, K.; Arakawa, H.; Ohga, Y.; Shinpo, A.; Sug, S. Chem. Commun. 2001, 569, indoline (Horiuchi, T.; Miura, H.; Sumioka, K.; Uchida, S. J. Am. Chem. Soc. 2004, 126, 12218) and merocyanine (Otaka, H.; Kira, M.; Yano, K.; Ito, S.; Mitekura, H.; Kawata, T.; Matsui, F. J. Photochem. Photobiol. A: Chem. 2004, 164, 67) are used for forming dye-sensitized solar cells.

In addition, US Patent Application Publication No. 20100122729 discloses a dye compound with better photoelectric conversion efficiency. However, the preparation of the organic-sensitized dye is complicate, and it is also hard to control the preparation condition. Japanese Patent No. 4442105 discloses a dye compound having a spacer structure for absorbing light and maintaining the photoelectric conversion efficiency.

The dye in the dye-sensitized solar cell is critical to the cell efficiency. Thus, it is important to develop a dye compound for increasing efficiency of the dye-sensitized solar cell. Further, it is also an urgent issue to simplify synthesis of a dye compound for lowering the cost of the solar cell.

### SUMMARY OF THE INVENTION

The present invention provides a novel dye compound for a dye-sensitized solar cell. The dye compound of the present invention has a structure of formula (I) or a salt thereof:

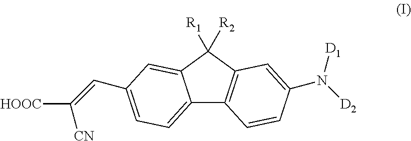

wherein R₁ and R₂ are, independently, H, C₁-C₁₂alkoxy or a halogen; and D₁ and D₂ are, independently, C₁-C₁₂alkyl, or D₁, D₂ and N form one of

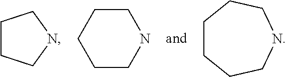

In one embodiment, R₁, R₂, D₁ and D₂ are all butyl groups.

In one embodiment, R₁ and R₂ are, independently, C₁-C₁₂alkyl, and D₁, D₂ and N form

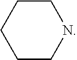

For example, R₁ and R₂ are both butyl groups, and D₁, D₂ and N form

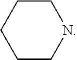

Alternatively, R₁ and R₂ are both ethyl groups, and D₁, D₂ and N form

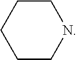

The dye compound of formula (I) may be

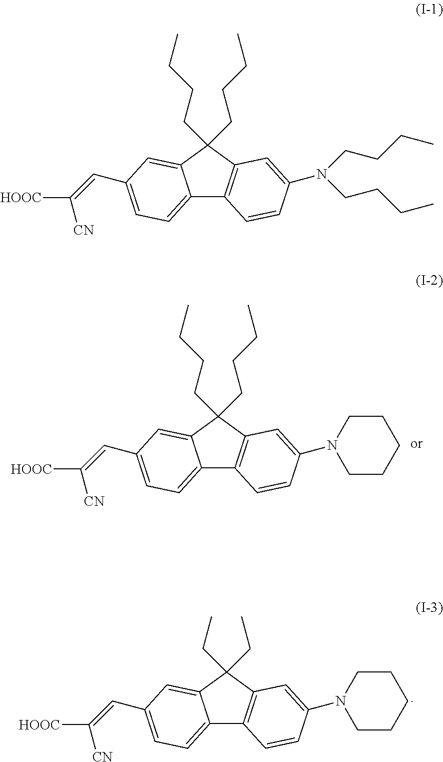

The dye compound is a free acid in embodiments of the present invention, but may be a salt, an alkali metal salt or a quaternary ammonium salt.

In addition, the dye compound is applicable to a dye-sensitized solar cell.

The present invention provides a simple synthesis method for forming a dye compound. In comparison with the prior art, the method is fewer in step and lower in cost, and is easy to be controlled.

The present invention further provides a dye-sensitized solar cell with higher photoelectric conversion efficiency. The dye-sensitized solar cell includes a photoanode having the dye compound of the present invention, a cathode and an electrolyte layer disposed between the photoanode and the cathode.

In the dye-sensitized solar cell of the present invention, the photoanode includes a transparent substrate, a transparent conductive film, a porous semiconductor film, and a dye compound, and the dye compound is the previously mentioned dye compound.

In the dye-sensitized solar cell of the present invention, the material of the transparent substrate is not limited, but needs to be transparent. Preferably, the transparent substrate is a great shelter from liquid or gas coming from outside of the solar cell and has great tolerance to solvents. For example, the transparent substrate may be, but not limited to, an inorganic substrate such as quartz or glass, or a transparent plastic substrate such as polyethylene terephthalate (PET), polyethylene naphthalate (PEN), polycarbonate (PC), poly(ethylene) (PE), polypropylene (PP) or polyimide (PI). In addition, there is no limitation to the thickness of the transparent substrate, and the selection of the transparent substrate may depend on transparency and requirements of the solar cell. Preferably, the transparent substrate is glass. In the dye-sensitized solar cell of the present invention, the transparent conductive film may be made of indium tin oxide (ITO), fluorine-doped tin oxide (FTO), ZnO-Ga₂O₃, ZnO-Al₂O₃, or a tin oxide.

In the dye-sensitized solar cell of the present invention, the porous semiconductor film is made of semiconductor porous particles. The semiconductor porous particle may include one or more of the group consisting of silicon, titanium dioxide, tin dioxide, zinc oxide, tungsten trioxide, niobium pentaoxide, and SrTiO₃. Preferably, the semiconductor porous particle is made of titanium dioxide. The average diameter of the semiconductor porous particle is 5 to 500 nm, and preferably 10 to 50 nm. The thickness of the porous semiconductor film is 5 to 25 micrometer.

The material of the cathode is not limited, but needs to be conductive. Alternatively, the cathode may include a body make of an insulation material and a conductive layer formed on the surface of the body facing the photoanode. The material with electrochemical stability such as Pt, Au, C, and the like may be used for forming the cathode.

The electrolyte layer of the dye-sensitized solar cell may include any substrate having electrons and/or holes.

In addition, the present invention provides a dye solution including 0.01 to 1 wt % of the dye compound of the present invention and 99 to 99.99 wt % of an organic solvent based on the total weight of the dye solution. The organic solvent is one selected from the group consisting of acetonitrile, methanol, ethanol, propanol, butanol, N,N-dimethylformamide, and N-methylpyrrolidone.

### BRIEF DESCRIPTION OF THE DRAWINGS

FIG. 1 is an NMR spectrum of the dye compound (I-1) of the present invention;

FIG. 2 is an NMR spectrum of the dye compound (I-2) of the present invention; and

FIG. 3 is an NMR spectrum of the dye compound (I-3) of the present invention.

### DETAILED DESCRIPTION OF THE PREFERRED EMBODIMENTS

The following specific examples are used for illustrating the present invention. A person skilled in the art can easily conceive the other advantages and effects of the present invention.

The dye compound of the present invention may be synthesized according to one of Scheme 1 to Scheme 3. In one embodiment, the dye compound of formula (I-1) was prepared according to Scheme 1.

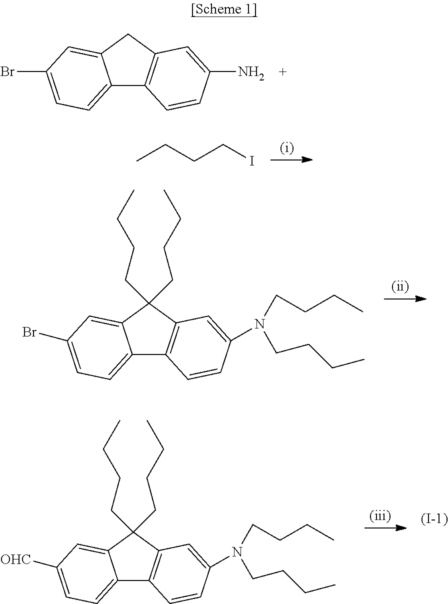

wherein
(i) represents potassium tert-butoxide, potassium carbonate, N,N-dimethylformamide, and dioxane
(ii) represents butyllithium, tetrahydrofuran, and N,N-dimethylformamide; and
(iii) represents cyanoacetic acid, piperidine, and acetonitrile.

As shown in Scheme 1, a reaction of 7-bromo-9H-fluoren-2-ylamine and n-butyl iodide was performed to form (7-bromo-9,9-di-n-butyl-9H-fluoren-2-yl)-di-n-butylamine. The formylation of (7-bromo-9,9-di-n-butyl-9H-fluoren-2-yl)-di-n-butylamine and N,N-dimethylformamide was performed to form 9,9-di-n-dibutyl-7-(di-n-butylamino)-9H-fluorene-2-carbaldehyde. Then, the reaction of 9,9-di-n-dibutyl-7-(di-n-butylamino)-9H-fluorene-2-carbaldehyde and cyanoacetic acid was performed in the presence of acetonitrile by using piperidine as a catalyst, so as to form 2-cyano-3-(9,9-di-n-butyl-7-di-n-butylamino-9H-fluoren-2-yl)acrylic acid (formula (I-1)).

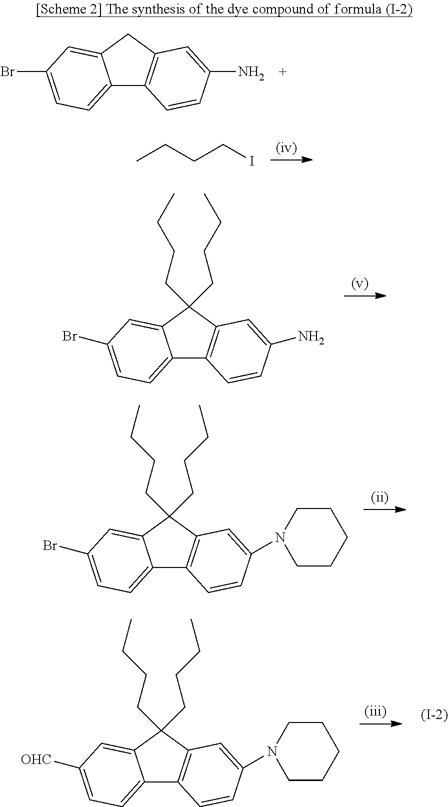

wherein
(iv) represents potassium tent-butoxide, and tetrahydrofuran; and
(v) represents potassium carbonate, and N,N-dimethylformamide.

As shown in Scheme 2, a reaction of 7-bromo-9H-fluoren-2-y-lamine and n-butyl iodide was performed to form 7-bromo-9,9-dibutyl-9H-fluoren-2-amine. The cyclization of 7-bromo-9,9-dibutyl-9H-fluoren-2-amine and 1,5-diiodopentane was performed to form 1-(7-bromo-9,9-dibutyl-9H-fluoren-2-yl)piperidine. The formylation of 1-(7-bromo-9,9-dibutyl-9H-fluoren-2-yl)piperidine and N,N-dimethylformamide was performed to form 9,9-dibutyl-7-(piperidin-1-yl)-9H-fluorene-2-carbaldehyde.

The reaction of 9,9-dibutyl-7-(piperidin-1-yl)-9H-fluorene-2-carbaldehyde and cyanoacetic acid was performed in the presence of acetonitrile by using piperidine as a catalyst, so as to form 2-cyano-3-(9,9-dibutyl-7-(piperidin-1-yl)-9H-fluoren-2-yl)acrylic acid (formula (I-2)).

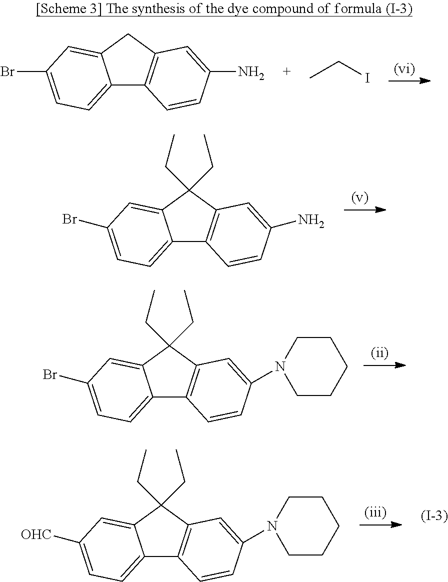

wherein
(iv) represents potassium tert-butoxide and tetrahydrofuran; and
(v) represents potassium carbonate and N,N-dimethylformamide.

As shown in Scheme 3, a reaction of 7-bromo-9H-fluoren-2-yl-amine and iodoethane was performed to form 7-bromo-9,9-diethyl-9H-fluoren-2-amine. Then, the cyclization of 7-bromo-9,9-diethyl-9H-fluoren-2-amine and 1,5-diiodopentan was performed to form 1-(7-bromo-9,9-diethyl-9H-fluoren-2-yl)piperidine. The reaction of 1-(7-bromo-9,9-diethyl-9H-fluoren-2-yl)piperidine and N,N-dimethylformamide was performed to form 9,9-diethyl-7-(piperidin-1-yl)-9H-fluorene-2-carbaldehyde.

The reaction of 9,9-diethyl-7-(piperidin-1-yl)-9H-fluorene-2-carbaldehyde and cyanoacetic acid was performed in the presence of acetonitrile by using piperidine as a catalyst, so as to form 2-cyano-3-(9,9-diethyl-7-(piperidin-1-yl)-9H-fluoren-2-yl)acrylic acid (formula (I-3)).

The fabrication of the dye-sensitized solar cell of the present invention may be a common method.

The material of the transparent substrate is not limited, but needs to be transparent. Preferably, the transparent substrate is a great shelter from liquid or gas coming from outside of the solar cell and has great tolerance to solvents. For example, the transparent substrate may be, but not limited to, an inorganic substrate such as quartz or glass, or a transparent plastic substrate such as polyethylene terephthalate (PET), polyethylene naphthalate (PEN), polycarbonate (PC), poly(ethylene) (PE), polypropylene (PP) or polyimide (PI). In addition, there is no limitation to the thickness of the transparent substrate, and the selection of the transparent substrate may depend on transparency and requirements of the solar cell. In one embodiment, the transparent substrate is glass.

The transparent conductive film may be made of indium tin oxide (ITO), fluorine-doped tin oxide (PTO), ZnO-Ga₂O₃, ZnO-Al₂O₃ or a tin oxide. In one embodiment, the transparent conductive film is made of tin oxide doped with fluoride.

The porous semiconductor film is made of semiconductor porous particles. The semiconductor porous particle may include one or more of the group consisting of silicon, titanium dioxide, tin dioxide, zinc oxide, tungsten trioxide, niobium pentaoxide and SrTiO₃. The semiconductor porous particles were prepared as a paste, and then applied on a transparent conductive substrate by wet coating such as blading, screen printing, spin-coating or spraying. Further, in order to obtain a proper thickness, the application of the paste may be performed for once or multiple times. The semiconductor film may be single layer or multiple layers, wherein the multiple layers are respectively formed from particles with different diameters. For example, semiconductor porous particles with 5-50 nm diameters were applied to form a semiconductor film layer with a thickness of 5-20 micrometer, and then semiconductor porous particles with 200-400 nm diameters were applied to form another semiconductor film layer with a thickness of 3-5 micrometer. After drying at 50-100° C., the multiple layers were sintered at 400-500° C. for 30 minutes.

The dye compound of the present invention may be dissolved in a proper solvent to form a dye solution. The proper solvent may be, but not limited to, acetonitrile, methanol, ethanol, propanol, butanol, N,N-dimethylformamide and N-methylpyrrolidone or a combination thereof. The transparent substrate coated with the semiconductor film was immersed into the dye solution to adsorb the dye, and then to be dried so as to obtain a photoanode of a dye-sensitized solar cell.

The material of the cathode is not limited, but needs to be conductive. Alternatively, the cathode may include a body made of an insulation material and a conductive layer formed on the surface of the body facing the photoanode. The material with electrochemical stability such as Pt, Au, C and the like may be used for forming the cathode.

The electrolyte layer of the dye-sensitized solar cell may include any substrate having electrons and/or holes. The electrolyte liquid for forming the electrolyte layer may be acetonitrile solution containing iodine, N-methylpyrrolidone solution containing iodine, or 3-methoxypropionitrile solution containing iodine. In one embodiment, the electrolyte liquid is acetonitrile solution containing iodine.

The formation of the dye-sensitized solar cell is illustrated as follows.

The paste having titanium oxide particles with 20-30 nm diameters was applied on a glass substrate coated with fluorine-doped tin oxide (FTO) by screen printing for once or several times, and then sintered at 450° C. for 30 minutes, allowing the coated fluorine-doped tin oxide (FTO) to be formed into the porous titanium oxide film on the glass substrate.

The dye compound was dissolved in a mixed solution having acetonitrile and t-butnaol (1:1 v/v) to form a dye solution. Then, the above glass substrate formed with the porous titanium oxide film was immersed in the dye solution to adsorb the dye, and then dried to form a photoanode.

The glass substrate coated with fluorine-doped tin oxide (FTO) was drilled to form an injection hole with 0.75 mm diameter for the electrolyte to be injected through. Then, the H₂PtCl₆ solution was applied on the glass substrate coated with fluorine-doped tin oxide (FTO), and then the glass substrate was heated to 400° C. for 15 minutes, so as to form a cathode.

The thermoplastic polymer film having a thickness of 60 micrometer was disposed between the photoanode and the cathode, and a pressure was applied on the two electrode at 120-140° C. to adhere the two electrodes.

The electrolyte solution (acetonitrile solution containing 0.03 M I₂/0.3 M LiI/0.5 M tert-butyl-pyridine) was injected into the injection hold, and then the injection hole was sealed with a thermoplastic polymer film, so as to obtain the dye-sensitized solar cell of the present invention.

The following embodiments are used for illustrating the present invention, and claims of the present invention are not limited to the following embodiments. The compound is illustrated as a free acid, but in practice may be a salt, an alkali metal salt or a quaternary ammonium salt. The temperature is demonstrated with centigrade, and parts and percentage are demonstrated by weight. The relationship of weight parts and volume parts is like the relationship of kilogram and liter.

According to Scheme I to Scheme 3, the detailed description of the dye compound of the present invention is as follows.

### Embodiment 1

### Synthesis of 2-cyano-3-(9,9-di-n-butyl-7-di-n-butylamino-9H-fluoren-2-yl)acrylic acid (formula (I-1))

0.52 part of 7-bromo-9H-fluoren-2-ylamine, 2.21 parts of n-butyl iodide, 0.67 part of potassium tert-butoxide and 0.83 part of potassium carbonate were mixed with 10 parts of dry N,N-dimethylformamide and 10 parts of 1,4-dioxane under nitrogen, and then the mixture was heated at 95° C. for 24 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 7-bromo-9,9-dibutyl-9H-fluoren-2-yl)-dibutylamine, which was light yellow liquid. The yield was 83%.

0.5 part of 7-bromo-9,9-dibutyl-9H-fluoren-2-yl-dibutylamin was sealed with nitrogen, added with and dissolved by tetrahydrofuran, and then the mixture was cooled down to −80° C. The mixture was added with 0.2 part of butyllithium and then added with 0.16 part of N,N-dimethylformamide. Then, the mixture was extracted with dichloromethane and dried over magnesium sulfate, and then the solvent was removed so as to obtain the crude product. The crude product was purified by silica gel column chromatography, and eluted with dichloromethane/hexane to obtain 9,9-di-n-dibutyl-7-(di-n-butylamino)-9H-fluorene-2-carbaldehyde, which was yellow solid. The yield was 50%.

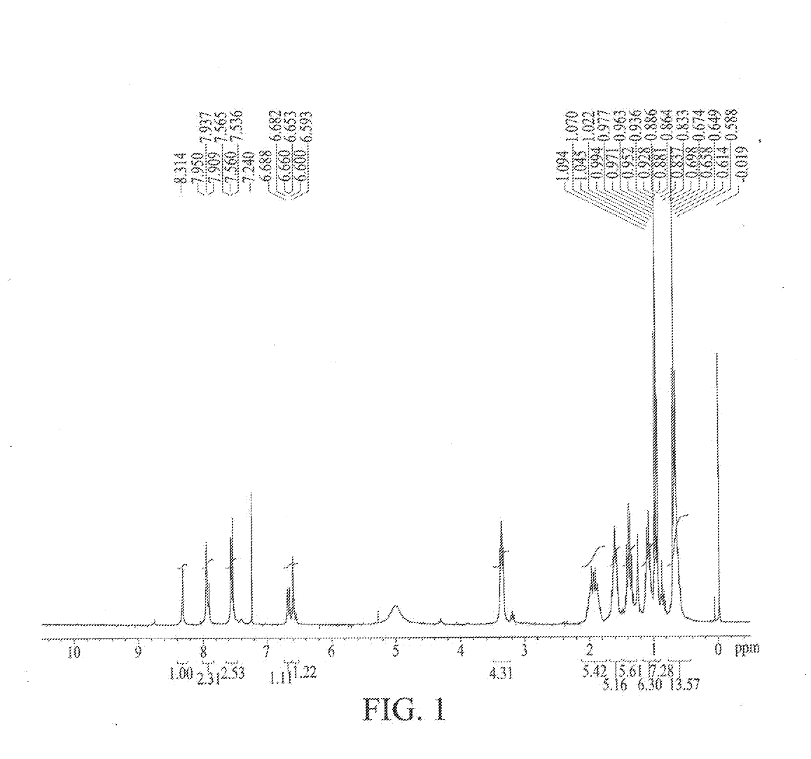

0.18 part of 9,9-di-n-dibutyl-7-(di-n-butylamino)-9H-fluorene-2-carbaldehyde, 0.05 part of cyanoacetic acid and 0.017 part of piperidine were added into 10 parts of acetonitrile, and mixed under nitrogen. The mixture was heated at 90° C. for 6 hours. Then, the mixture was cooled down to room temperature, and filtered to obtain the solid. The solid was washed with water, ether and acetonitrile in sequence, so as to obtain a dark red solid. This solid was purified by silica gel column chromatography, and eluted with dichloromethane/hexane to obtain the compound, which was dark red. The yield was 73%. The NMR spectrum of the product was shown in FIG. 1.

### Embodiment 2

### Synthesis of 2-cyano-3-(9,9-dibutyl-7-(piperidin-1-yl)-9H-fluoren-2-yl)acrylic acid (formula (I-2))

1.44 parts of 7-bromo-9H-fluoren-2-yl-amine, 5.52 parts of n-butyl iodide and 3.37 parts of potassium tert-butoxide were mixed with 50 parts of dry tetrahydrofuran under nitrogen, and then the mixture was heated at 50° C. for 18 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 7-bromo-9,9-dibutyl-9H-fluoren-2-amine, which was black liquid. The yield was 76%.

0.18 part of 7-bromo-9,9-dibutyl-9H-fluoren-2-amine was sealed with nitrogen, and 0.20 part of potassium carbonate added with and dissolved by 10 parts of dry N,N-dimethylformamide, and then added with 0.19 part of 1,5-diiodopentane for cyclization. The mixture was heated at 120° C. for 24 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 1-(7-bromo-9,9-dibutyl-9H-fluoren-2-yl)piperidine, which was black liquid. The yield was 70%.

0.15 part of 1-(7-bromo-9,9-dibutyl-9H-fluoren-2-yl)piperidin was sealed with nitrogen, added with and dissolved by tetrahydrofuran, and then the mixture was cooled down to −80° C. The mixture was added with 0.06 part of butyllithium and then added with 0.05 part of N,N-dimethylformamide. Then, the mixture was extracted with dichloromethane and dried over magnesium sulfate, and then the solvent was removed so as to obtain the crude product. The crude product was purified by silica gel column chromatography, and eluted with dichloromethane/hexane to obtain 9,9-diethyl-7-(piperidin-1-yl)-9H-fluorene-2-carbaldehyde, which was yellow solid. The yield was 54%.

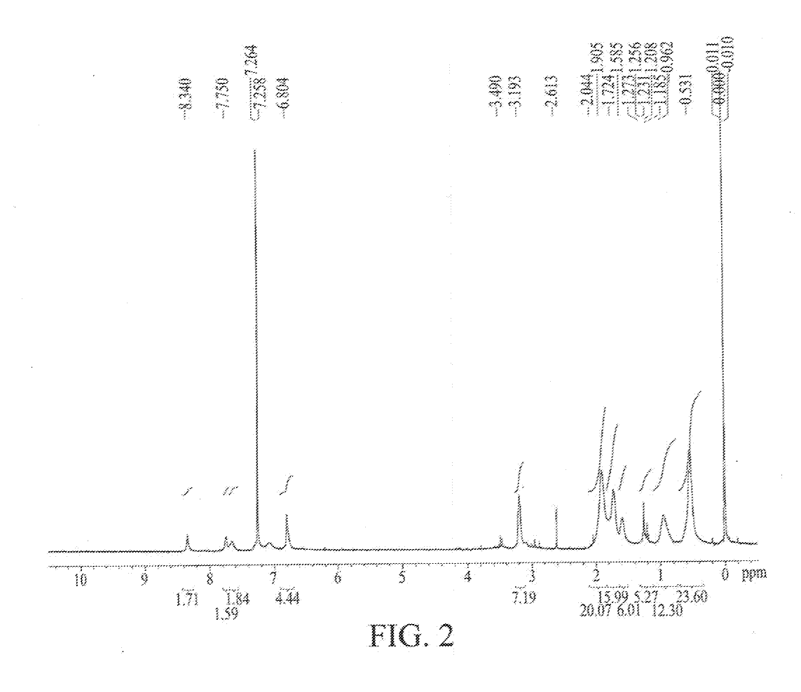

0.07 part of 9,9-diethyl-7-(piperidin-1-yl)-9H-fluorene-2-carbaldehyde, 0.02 part of cyanoacetic acid and 0.017 part of piperidine were added into 5 parts of acetonitrile, and mixed under nitrogen. The mixture was heated at 90° C. for 6 hours. Then, the mixture was cooled down to room temperature, and filtered to obtain the solid. The solid was washed with water, ether and acetonitrile in sequence, so as to obtain a dark red solid. This solid was purified by silica gel column chromatography, and eluted with dichloromethane/hexane to obtain the compound, which was dark red. The yield was 73%. The NMR spectrum of the product was shown in FIG. 2.

### Embodiment 3

### Synthesis of 2-cyano-3-(9,9-diethyl-7-(piperidin-1-yl)-9H-fluoren-2-yl)acrylic acid (formula (I-3))

1.44 parts of 7-bromo-9H-fluoren-2-ylamine, 7.79 parts of iodoethane and 3.37 parts of potassium tert-butoxide were mixed with 50 parts of dry tetrahydrofuran under nitrogen, and then the mixture was heated at 50° C. for 18 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 7-bromo-9,9-diethyl-9H-fluoren-2-amine, which was black liquid. The yield was 76%.

0.31 part of 7-bromo-9,9-diethyl-9H-fluoren-2-amine sealed with nitrogen and 0.13 part of potassium carbonate were added with and dissolved by 10 parts of dry N,N-dimethylformamide, and then added with 0.32 part of 1,5-diiodopentane for cyclization. The mixture was heated at 120° C. for 24 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 1-(7-bromo-9,9-diethyl-9H-fluoren-2-yl)piperidine, which was light yellow brown solid. The yield was 65%.

0.16 part of 1-(7-bromo-9,9-diethyl-9H-fluoren-2-yl)piperidine sealed with nitrogen was added with and dissolved by tetrahydrofuran. Then, the mixture was cooled down to −80° C. The mixture was added with 0.07 part of butyllithium and then added with 0.05 part of N,N-dimethylformamide. Then, the mixture was extracted with dichloromethane and dried over magnesium sulfate, and then the solvent was removed so as to obtain the crude product. The crude product was purified by silica gel column chromatography, and eluted with dichloromethane/hexane to obtain 9,9-diethyl-7-(piperidin-1-yl)-9H-fluoren-2-carbaldehyde, which was yellow solid. The yield was 30%.

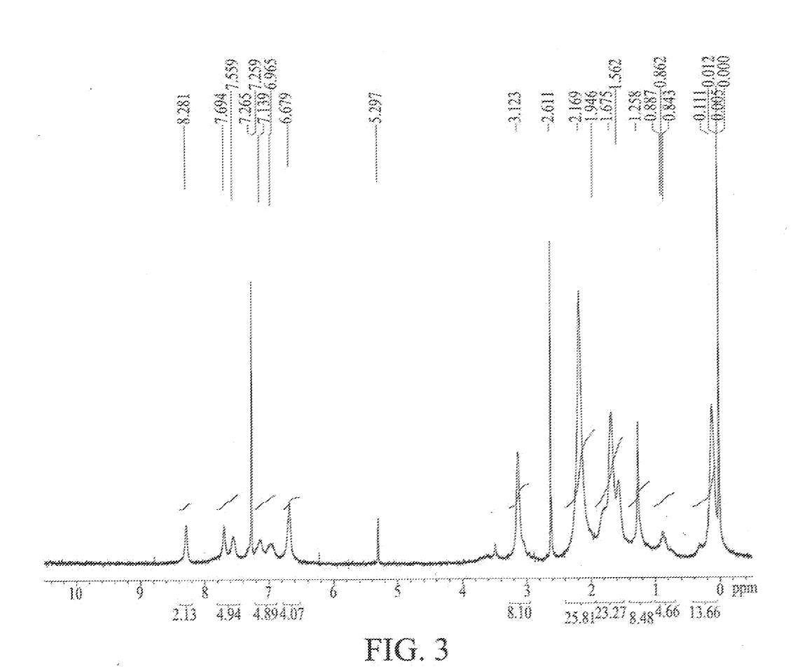

0.09 part of 9,9-diethyl-7-(piperidin-1-yl)-9H-fluoren-2-carbaldehyde, 0.01 part of cyanoacetic acid and 0.017 part of piperidine were added into 5 parts of acetonitrile, and mixed under nitrogen. The mixture was heated at 90° C. for 6 hours. Then, the mixture was cooled down to room temperature, and filtered to obtain the solid. The solid was washed with water, ether and acetonitrile in sequence, so as to obtain a dark red solid. This solid was purified by silica gel column chromatography, and eluted with dichloromethane/hexane to obtain the compound, which was dark red. The yield was 70%. The NMR spectrum of the product was shown in FIG. 3.

### Embodiment 4

### Formation of the Dye-Sensitized Solar Cell

The paste having titanium dioxide particles with 20-30 nm diameters was applied on a glass plate coated with fluorine-doped tin oxide (FTO) (thickness: 4 mm, resistance: 10Ω), and sintered at 450° C. for 30 minutes to form the porous titanium dioxide film with thickness of 10-12 μm.

The dye compound of Embodiment 1 was dissolved in the solution containing acetonitrile and t-butanol (1:1 v/v) to form 0.5 M dye solution. The above glass plate with the porous titanium dioxide film was immersed in the dye solution to adsorb the dye for 16-24 hours, and then dried to obtain a photoanode.

The glass plate coated with fluorine-doped tin oxide (FTO) was drilled to form an injection hole with 0.75 mm diameter for the electrolyte to be injected through. Then, the H₂PtCl₆ solution was applied on the glass plate coated with fluorine-doped tin oxide (FTO), and then the glass substrate was heated to 400° C. for 15 minutes, so as to form a cathode.

The thermoplastic polymer film having a thickness of 60 micrometer was disposed between the photoanode and the cathode, and a pressure was applied on the two electrode at 120-140° C. to adhere the two electrodes.

The electrolyte solution (acetonitrile solution containing 0.03 M I₂/0.3 M LiI/0.5 M 4-tert-butyl-pyridine) was injected, and then the injection hole was sealed with the thermoplastic polymer film, so as to obtain the dye-sensitized solar cell of the present invention.

### Comparative Example 1

### Synthesis of the Dye Compound Disclosed in Japanese Patent No. 4442105 and the Dye-Sensitive Solar Cell

1 part of 2,7-bromo-9H-fluorene, 1.06 parts of iodomethane and 0.83 part of potassium tert-butoxide were added and mixed with 10 parts of dry tetrahydrofuran under nitrogen. The mixture was heated at 25° C. for 18 hours. Then, the mixture was cooled down, extracted with ether and dried over magnesium sulfate, and then the solvent was removed so as to obtain the crude product. The crude product was purified by silica gel column chromatography, and eluted with dichloromethane/hexane to obtain 2,7-bromo-9,9-methyl-9H-fluorene, which was a yellow solid. The yield was 85%.

1.2 parts of 2,7-bromo-9,9-methyl-9H-fluorene, 0.32 part of diphenylamine, 0.27 part of sodium tert-butoxide and catalysts (0.021 part of Pd(dba)₂ bis(dibenzylideneacetone)palladium(0) and 0.021 part of dppf (1,1′-bis(diphenylphosphino)ferrocene) were added and mixed with 5 parts of toluene. The mixture was heated at 80° C. for 24 hours. Then, the mixture was filtered with celite, and then the solvent was removed to obtain the residue. The residue was purified by silica gel column chromatography, and eluted with dichloromethane/hexane to obtain 7-bromo-9,9-dimethyl-N,N-diphenyl-9H-fluoren-2-amine, which is a yellow solid. The yield was 55%.

0.35 part of 7-bromo-9,9-dimethyl-N,N-diphenyl-9H-fluoren-2-amine was added with and dissolved by 30 parts of tetrahydrofuran. The mixture was cooled down to −80° C. The mixture was added with 1.05 parts of butyllithium and then added with 0.7 part of N,N-dimethylformamide. Then, the mixture was extracted with dichloromethane and dried over magnesium sulfate, and then the solvent was removed so as to obtain the crude product. The crude product was purified by silica gel column chromatography, and eluted with dichloromethane/hexane to obtain 7-(diphenylamino)-9,9-methyl-9H-fluoren-2-carbaldehyde, which was a yellow solid. The yield was 50%.

7-(diphenylamino)-9,9-methyl-9H-fluoren-2-carbaldehyde, 0.05 part of cyanoacetic acid and 0.017 part of piperidine were added into 8 parts of acetonitrile, and mixed under nitrogen. The mixture was heated at 90° C. for 6 hours. Then, the mixture was cooled down to room temperature, and filtered to obtain the solid. The solid was washed with water, ether and acetonitrile in sequence, so as to obtain a dark red solid. This solid was purified by silica gel column chromatography, and eluted with dichloromethane/methanol to obtain 2-cyano-3-(7-(diphenylamino)-9,9-methyl-9H-fluoren-2-yl)acrylic acid (formula (II)), which was dark red. The yield was 50%.

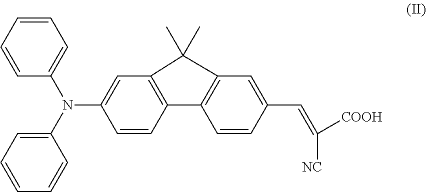

Formation of the Dye-Sensitized Solar Cell

The steps were similar to those in Embodiment 4 except that the dye compound of Embodiment 1 was replaced with the dye compound (II) of Comparative Example 1.

### Comparative Example 2

### Synthesis of the Dye Compound Disclosed in Us Patent Application Publication No. 20100122729 and the Dye-Sensitive Solar Cell

0.52 part of 7-bromo-9H-fluoren-2-amine, 2.21 parts of n-butyl iodide, 0.67 part of potassium tert-butoxide and 0.83 part of potassium carbonate were added and mixed with 10 parts of dry N,N-dimethylformamide and 10 parts of 1,4-dioxane under nitrogen. The mixture was heated at 95° C. for 24 hours. Then, the mixture was cooled down, extracted with ether and dried over magnesium sulfate, and then the solvent was removed so as to obtain the residue. The residue was purified by silica gel column chromatography, and eluted with dichloromethane/hexane to obtain 7-bromo-9,9-dibutyl-9H-fluoren-2-yl)-dibutylamine, which is light yellow liquid. The yield was 83%.

0.49 part of 7-bromo-9,9-dibutyl-9H-fluoren-2-yl)-dibutylamine, 0.19 part of 5-formyl-2-thiopheneboronic acid, 0.41 part of potassium carbonate and 0.16 part of PdCl₂(dppf), [1,1′-bis(diphenylphosphino)ferrocene]dichloropalladium(II), were added and mixed with 5 parts of toluene and 5 parts of methanol under nitrogen. The mixture was heated at 60° C. for 18 hours. Then, the mixture was cooled down, extracted with ether and dried over magnesium sulfate, and then the solvent was removed so as to obtain the residue. The residue was purified by silica gel column chromatography, and eluted with dichloromethane/hexane to obtain 5-(9,9-dibutyl-7-dibutylamino-9H-fluoren-2-yl)-thiophene-2-carbaldehyde, which is an orange red solid. The yield was 61%.

0.23 part of 5-(9,9-dibutyl-7-dibutylamino-9H-fluoren-2-yl)-thiophene-2-carbaldehyde, 0.05 part of cyanoacetic acid and 0.017 part of piperidine were added into 10 parts of acetonitrile, and mixed under nitrogen. The mixture was heated at 90° C. for 6 hours. Then, the mixture was cooled down to room temperature, and filtered to obtain a solid. The solid was washed with water, ether and acetonitrile in sequence, so as to obtain a dark red solid. This solid was purified by silica gel column chromatography, and eluted with dichloromethane/methanol to obtain

2-cyano-3-[5-(9,9-dibutyl-7-dibutylamino-9H-fluoren-2-yl)-thiophen-2-yl]-acrylic acid (formula (III)), which was a dark red solid. The yield was 73%.

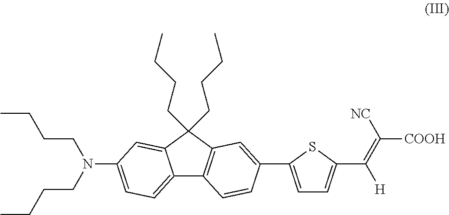

Formation of the Dye-Sensitized Solar Cell

The steps were similar to those in Embodiment 4 except that the dye compound of Embodiment 1 was replaced with the dye compound (III) of Comparative Example 2.

Test of Photoelectric Efficiency

The open circuit voltage (V_{oc}), short circuit current (J_{sc}), fill factors (FF) and photoelectric conversion efficiency (η) of the dye-sensitized solar cells of Embodiment 1, Comparative Example 1 and Comparative Example 2 were tested at AM 1.5 illumination.

The test results of the dye compounds and the dye-sensitized solar cells were shown in Table 1.

TABLE 1

| | Dye | | J_{sc} | | |
|---|---|---|---|---|---|
| | compound | V_{oc} (V) | (mA/cm²) | FF | (%) |
| Embodiment 1 | (I-1) | 0.84 | 8.09 | 69.67 | 4.76 |
| Comparative | (II) | 0.88 | 7.18 | 72.04 | 4.54 |
| Example 1 | | | | | |
| Comparative | (III) | 0.73 | 9.48 | 67.44 | 4.55 |
| Example 2 | | | | | |

It is shown in Table 1 that the dye-sensitized solar cell using the dye compound of the present invention has outstanding photoelectric properties.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

What is claimed is:

1. A dye compound having a structure of formula (I) or a salt thereof: 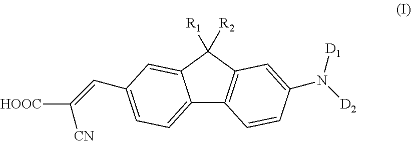
wherein R₁ and R₂ are, independently, H, C₁-C₁₂alkyl, C₁-C₁₂alkoxy or a halogen; and D₁ and D₂ are, independently, C₁-C₁₂alkyl, or D1, D2 and N form one of

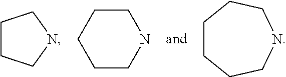

2. The dye compound of claim 1, wherein R₁, R₂, D₁ and D₂ are all butyl groups.

3. The dye compound of claim 1, wherein R₁ and R₂ are, independently, C₁-C₁₂alkyl, and D₁, D₂ and N form

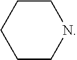

4. The dye compound of claim 3, wherein R₁ and R₂ are both butyl groups, and D₁, D₂ and N form

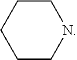

5. The dye compound of claim 3, wherein R₁ and R₂ are both ethyl groups, and D₁, D₂ and N form

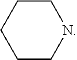

6. A dye-sensitized solar cell, comprising:
a photoanode, comprising a dye compound having a structure of formula (I) or a salt thereof, 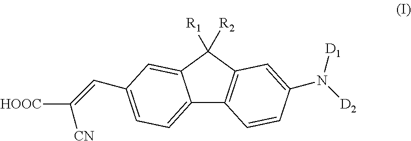
wherein R₁ and R₂ are, independently, H, C₁-C₁₂alkyl, C₁-C₁₂alkoxy or a halogen; and D₁ and D₂ are, independently, C₁-C₁₂alkyl, or D1, D2 and N form one of 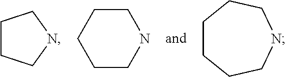
a cathode; and
an electrolyte layer disposed between the photoanode and the cathode.

7. The dye-sensitized solar cell of claim 6, wherein R₁, R₂, D₁ and D₂ are all butyl groups.

8. The dye-sensitized solar cell of claim 6, wherein R₁ and R₂ are both butyl groups, and D₁, D₂ and N form

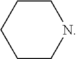

9. The dye-sensitized solar cell of claim 6, wherein R₁ and R₂ are both ethyl groups, and D₁, D₂ and N form

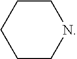

10. A dye solution, comprising:
a dye compound represented by the following formula (I), or a salt thereof, 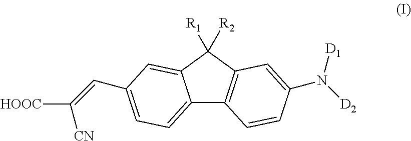
wherein an amount of the dye compound or the salt thereof is 0.01 to 1 wt % of the total weight of the dye solution; and
an organic solvent, wherein an amount of the organic solvent is 99 to 99.99 wt % of the total weight of the dye solution, and the organic solvent is one selected from the group consisting of acetonitrile, methanol, ethanol, propanol, butanol, N,N-dimethylformamide and N-methylpyrrolidone.
